(19) United States
(12) Patent Application Publication
(10) Pub. No.: US 2024/0400517 A1
(43) Pub. Date: Dec. 5, 2024

(54) CHIRAL BIMETALLIC COOPERATIVE CATALYSIS SYSTEM AND USE THEREOF IN ASYMMETRIC SYNTHESIS OF BEDAQUILINE

(71) Applicant: SHANGHAI JIAO TONG UNIVERSITY, Shanghai (CN)

(72) Inventors: Wanbin ZHANG, Shanghai (CN); Feng GAO, Shanghai (CN); Jing LI, Shanghai (CN); Tanveer AHMAD, Shanghai (CN); Zhenfeng ZHANG, Shanghai (CN); Qianjia YUAN, Shanghai (CN)

(73) Assignee: SHANGHAI JIAO TONG UNIVERSITY, Shanghai (CN)

(21) Appl. No.: 18/699,602

(22) PCT Filed: Mar. 17, 2023
(86) PCT No.: PCT/CN2023/082161

§ 371 Date: Apr. 9, 2024

(30) Foreign Application Priority Data
Feb. 8, 2022 (CN) 202210119308.2
Jun. 27, 2022 (CN) 202210737425.5

(51) Int. Cl. C07D 215/227; B01J 23/04

(52) U.S. Cl. CPC C07D 215/227; B01J 23/04

(57) ABSTRACT

A chiral bimetallic cooperative catalysis system and use thereof in asymmetric synthesis of bedaquiline are provided. Specifically, the chiral bimetallic cooperative catalysis system is formed by a metallic lithium, sodium or potassium salt and another metal salt under the action of a suitable ligand and an additive. By means of the chiral bimetallic cooperative catalysis system, an addition reaction of 6-bromo-3-benzyl-2-methoxyquinoline (I) and 3-dimethylamino-1-naphthyl-1-propanone (II) is promoted, the selectivity is regulated, and a target product, (1R,2S)-bedaquiline, with high yield and high selectivity is obtained for the first time.

### CROSS REFERENCE TO THE RELATED APPLICATIONS

This application is the national phase entry of International Application No. PCT/CN2023/082161, filed on Mar. 17, 2023, which is based upon and claims priority to Chinese Patent Applications No. 202210119308.2, filed on Feb. 8, 2022, and and No. 202210737425.5, filed on Jun. 27, 2022, the entire contents of which are incorporated herein by reference.

### TECHNICAL FIELD

The present invention belongs to the technical field of asymmetric synthesis, relates to a chiral bimetallic cooperative catalysis system and use thereof in asymmetric synthesis of bedaquiline, in particular to establishment of a chiral bimetallic cooperative catalysis system and use of the system, and specifically relates to asymmetric synthesis of (1R,2S)-bedaquiline as an anti-tuberculosis drug.

### BACKGROUND

Tuberculosis (TB) is a disease with highest mortality caused by single pathogenic bacteria in the world. The number of new cases in China is nearly 900,000 every year, accounting for 9% of the number of new cases in the global world every year, and epidemic situations of drug-resistant tuberculosis are extremely serious and are higher than the average level in the global world. Bedaquiline, as a diarylquinoline compound, has been used as a first new anti-tuberculosis drug in the past fifty years. In 2012, the bedaquiline was approved by the Food and Drug Administration (FDA) for marketing in the United States, which was the first new drug approved for treatment of multi-drug resistant tuberculosis. In 2016, the bedaquiline was officially approved by the State Food and Drug Administration of China for marketing in China. The bedaquiline has four optical isomers, and only a (1R,2S) configuration is effectively used as a drug. At present, bedaquiline molecules with single configurations can be obtained only by resolution with chiral reagents in an industrial production process. Such methods have the problems of poor selectivity of a C—C bond during formation of addition products, obtaining of target products as secondary separation products, low synthesis efficiency and the like. Accordingly, such methods have a low yield and a high cost. Improvement of synthesis methods to reduce the production cost and make drugs affordable for low-income persons is a problem to be solved urgently by drug developers at home and abroad.

According to a synthesis patent of (1R,2S)-bedaquiline (patent authorization No. CN101180302B) in original research, a one-step synthesis method (FIG. 1) is disclosed. The method includes subjecting 6-bromo-3-benzyl-2-methoxyquinoline (I) to deprotonation at a benzyl site with lithium diisopropylamide (IDA) at a low temperature (−72° C. to −78° C.), followed by addition with 3-dimethylamino-1-naphthyl-1-propanone (II) to generate a mixture of 4 optical isomers of bedaquiline; and subjecting reaction materials to concentration, treatment with ethanol and resolution with (R)-(−)-1, l′-binaphthyl-2,2′-diyl hydrogenphosphate as a chiral resolving agent to obtain (1R,2S)-bedaquiline (A) with a low total yield of only 7-9%. In addition, efficient catalytic asymmetric synthesis has attracted the attention of scientists. A method for construction of first carbon chirality by asymmetric catalysis and construction of second carbon chirality by asymmetric synthesis has been reported by Shibasaki (J. Am. Chem. Soc. 2010, 132, 7905-7907) and Chandrasekhar (Eur. J. Org. Chem. 2011, 2057-2061) separately. This route has the problems of many reaction steps, low yield, high manufacturing cost and the like. According to a patent (patent authorization No. CN10686652A), the proportion of a (1R,2S)-bedaquiline optical isomer in a reaction product is increased under the action of a chiral inducer, and the product has an enantiomeric excess (ee) value of 90% and a diastereoselectivity (dr) value of 4:1, but results are still unsatisfactory. In addition, according to a research group in South Africa, the diastereoselectivity of bedaquiline (BDQ) was increased to 9:1 by a C₂-symmetric chiral amine ligand (ACS Omega 2020, 5, 3607-3611) in 2020, and then, an optically pure target compound was obtained by chiral resolution. However, the problem of enantioselectivity of a target product obtained by the method is still unsolved. Therefore, a more efficient asymmetric synthesis method for obtaining the target product (1R,2S)-bedaquiline is required to be developed.

### SUMMARY

In view of the above disadvantages of the prior art, the present invention aims to provide a chiral bimetallic cooperative catalysis system and use thereof in asymmetric synthesis of bedaquiline, and specifically relates to a chiral bimetallic cooperative catalysis system and use of the system in asymmetric synthesis of (1R,2S)-bedaquiline as an anti-tuberculosis drug.

The purposes of the present invention are realized through the following technical schemes.

In a first aspect, the present invention relates to a chiral bimetallic cooperative catalysis system for synthesis of (1R,2S)-bedaquiline. The chiral bimetallic cooperative catalysis system is formed by using a lithium, sodium or potassium salt in cooperation with another metal salt and selecting a suitable ligand and an additive. In the chiral bimetallic cooperative catalysis system, the suitable ligand is used for controlling metal ions. The system can efficiently promote the synthesis of (1R,2S)-bedaquiline.

As an embodiment, the lithium, sodium or potassium salt is derived from a combination of one or more of n-butyllithium, sec-butyllithium, tert-butyllithium, lithium diisopropylamide, lithium tetramethylpiperidine, lithium bis(trimethylsilyl)amide, sodium bis(trimethylsilyl)amide and potassium bis(trimethylsilyl)amide.

As an embodiment, the another metal salt is a metal halide, and the metal is Mg, Zn, Cu, Al, Ni, Co, Fe, Na, K, Ti, Ca or Li. Specifically, the another metal salt includes one or more of MgCl₂, ZnCl₂, CuCl₂, AlCl₃, NiCl₂, CoCl₂, FeCl₃, NaCl, KCl, TICl₃, CaCl₂, LiCl, LiBr, Lil and LiF.

As an embodiment, the ligand has a structural formula of

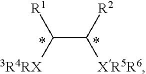

wherein X and X′ are separately selected from an N or O) atom, (that is to say, the X and the X′ may be a nitrogen atom or an oxygen atom simultaneously, or a nitrogen atom and an oxygen atom, respectively); R¹, R², R¹, R⁴, R′ and R′ are separately selected from H, C₁₋₁₆ straight-chain or branch-chain alkyl, aryl or C₃₋₇ cycloalkyl, the aryl is phenyl substituted with R⁷, the R⁷ is H, C₁₋₆ alkyl, halogen, C₁₋₆ alkoxyl or OCOR⁸ (the halogen is F, Cl, Br or I), the R⁸ is C₁₋₆ alkyl, the substituted with R⁷ refers to single substitution or multiple substitutions, and the multiple substitutions are the same or different; and the R¹, the R², the R³, the R⁴, the R⁵ and the R⁶ are the same or different or any two of the groups form a ring.

As an embodiment, in the structural formula of the ligand, the R¹ and the R² are the same or different or form a ring; the R³ and the R⁴ are the same or different or form a ring; the R³ and the R¹ form a ring or do not form a ring; the R⁵ and the R⁶ are the same or different or form a ring; the R⁵ and the R² form a ring or do not form a ring; and * refers to chirality with a configuration of RR, SS, RS or SR.

As an embodiment, the additive is an organic alkali with a structural formula of

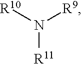

wherein R⁹, R¹⁰ and R¹¹ are separately H. C₁₋₁₆ straight-chain or branch-chain alkyl, aryl or C₃₋₇ cycloalkyl, the aryl is phenyl substituted with R¹², and the R¹² is H, C₁₋₆ alkyl, halogen, C₁₋₆ alkoxyl or OCOR¹¹ (the halogen is F, Cl, Br or I); the R¹³ is C₁₋₆ alkyl; the substituted with R¹² refers to single substitution or multiple substitutions, and the multiple substitutions are the same or different; and the R⁹, the R¹⁰ and the R¹¹ are the same or different or any two of the groups form a ring.

As an embodiment, the organic alkali is selected from 1,8-diazabicyclo[5.4.0]undec-7-ene (DBU), 1,5-diazobicyclo[4.3.0]non-5-ene (DBN), N,N,N′,N′-tetramethylethylenediamine (TMEDA), hexamethylphosphoric triamide (HMPA), 4-dimethylaminopyridine (DMAP), triethylenediamine (DABCO), 2,2,6,6-tetramethylpiperidine (TMP), N,N-diisopropylamine (DIPA), pyridine, pyrrolidine, amantadine, Sparteine and/BuNHSi (CH)₃.

In a second aspect, the present invention further relates to use of the chiral bimetallic cooperative catalysis system in synthesis of (1R,2S)-bedaquiline. In an organic solution of a chiral bimetallic cooperative catalysis system formed by a mixed reaction of a lithium, sodium or potassium salt, another metal salt, a ligand and an additive at a temperature of −78° C. to 0° C., an organic solution of 6-bromo-3-benzyl-2-methoxyquinoline (I) is slowly added dropwise to carry out a reaction, then an organic solution of 3-N,N-dimethylamino-1-naphthyl-1-propanone (II) is continuously added to carry out another reaction, and a resulting product is subjected to separation and purification to obtain (1R,2S)-bedaquiline.

As an embodiment, the mixed reaction is carried out for 1 min to 2 h. The time may be 1-10 min. 10-20 min, 20-30 min, 30-40 min, 40-50 min, 50-60 min. 1-1.5 h, 1.5-2 h and the like.

As an embodiment, the organic solution of 6-bromo-3-benzyl-2-methoxyquinoline (I) is added dropwise to carry out a reaction for 10 min to 12 h. The time may be 10-30 min. 30-50 min, 50-60 min, 1-1.5 b, 1.5-2 h, 2-2.5 h, 2.5-3 h, 3-3.5 h, 3.5-4 h, 4-4.5 h, 4.5-5 h, 5-5.5 h, 5.5-6 h, 6-7 h, 7-8 h, 8-9 h, 9-10 h, 10-11 h, 11-12 h and the like.

As an embodiment, the organic solution of 3-N,N-dimethylamino-1-naphthyl-1-propanone (II) is added to carry out another reaction for 10 min to 12 h. The time may be 10-30 min, 30-50 min, 50-60 min, 1-1.5 h, 1.5-2 h, 2-2.5 h, 2.5-3 h, 3-3.5 h, 3.5-4 h, 4-4.5 h, 4.5-5 h, 5-5.5 h, 5.5-6 h, 6-7 h, 7-8 h, 8-9 h, 9-10 h, 10-11 h, 11-12 h and the like.

As an embodiment, the use amount of the lithium, sodium or potassium salt is 1-5 times of the equivalent of the 6-bromo-3-benzyl-2-methoxyquinoline (I). The use amount may be 1-1.5 times of the equivalent, 1.5-2 times of the equivalent, 2-2.5 times of the equivalent, 2.5-3 times of the equivalent, 3-3.5 times of the equivalent, 3.5-4 times of the equivalent, 4-4.5 times of the equivalent or 4.5-5 times of the equivalent.

As an embodiment, the use amount of the another metal salt is 0.01-2.0 times of the equivalent of the 3-dimethylamino-1-naphtbyl-1-propanone (II). The use amount may be 0.01-0.1 times of the equivalent, 0.1-0.5 times of the equivalent, 0.5-1 times of the equivalent, 1-1.5 times of the equivalent or 1.5-2 times of the equivalent.

As an embodiment, the molar ratio of the metal salt to the chiral ligand is 1.0: (0.1-5.0). The molar ratio may be 1:0.1, 1:0.2, 1:0.5, 1:1, 1:1.2, 1:1.5, 1:2, 1:2.5, 1:3, 1:3.5, 1:4, 1:4.5 or 1:5.

As an embodiment, the use amount of the additive is 0.1-20 times of the equivalent of the 6-bromo-3-benzyl-2-methoxyquinoline (I). The use amount may be 0.1-0.5 times of the equivalent, 0.5-1 times of the equivalent, 1-1.5 times of the equivalent, 1.5-2 times of the equivalent, 2-5 times of the equivalent, 5-10 times of the equivalent, 10-15 times of the equivalent or 15-20 times of the equivalent.

As an embodiment, an organic solvent used in the organic solution is selected from a mixture of one or more of ethyl ether, tetrahydrofuran, methyl tert-butyl ether, 1,4-dioxane, diisopropyl ether and ethylene glycol dimethyl ether as well as toluene, xylene, ethylbenzene, n-hexane, cyclohexane, n-heptane and n-pentane.

The present invention has the following beneficial effects.

In current reports, the highest ratio of two pairs of diastereomers, (A+A′) and (B+B′), is 9:1, and the target product (1R,2S)-bedaquiline has an optimal ee value of 90%. However, in the present invention, the ratio of the two pairs of diastereomers, (A+A′) and (B+B′), can be as high as 10:1 under the premise that the enantioselectivity of the target product (1R,2S)-bedaquiline is as high as 99%.

### BRIEF DESCRIPTION OF THE DRAWINGS

Other features, purposes and advantages of the present invention will become more apparent by reading the detailed description of non-restrictive embodiments with reference to accompanying drawings below.

FIG. 1 is a schematic diagram showing synthesis of bedaquiline in one step disclosed by CN101180302B;

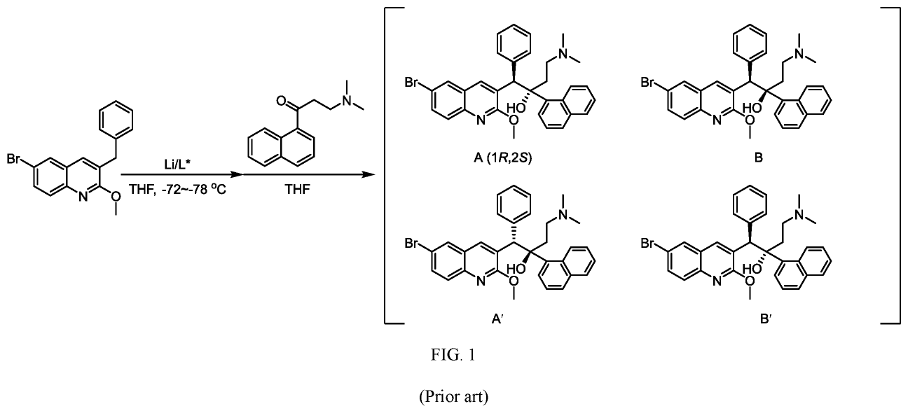

FIG. 2 is a schematic diagram showing asymmetric synthesis of bedaquiline in the present invention;

FIG. 3 shows hydrogen nuclear magnetic resonance spectra of a product (1R,2S)-bedaquiline;

FIG. 4 shows hydrogen nuclear magnetic resonance spectra of a product with a dr value of 4:1;

FIG. 5 shows hydrogen nuclear magnetic resonance spectra of a product with a dr value of 7:1;

FIG. 6 shows hydrogen nuclear magnetic resonance spectra of a product with a dr value of 10:1;

FIG. 7 shows hydrogen nuclear magnetic resonance spectra of a product with a dr value of 20:1; and

FIG. 8 shows liquid chromatography spectra of a product with an ee value of 99%.

### DETAILED DESCRIPTION OF THE EMBODIMENTS

The present invention is described in detail in combination with specific embodiments below. The following embodiments will facilitate the present invention further understood by persons skilled in the art, and are not intended to limit the present invention in any manner. It is to be noted that for persons of ordinary skill in the art, various modifications or improvements can be made without departing from the concept of the present invention. All the modifications or improvements fall within the scope of protection of the present invention.

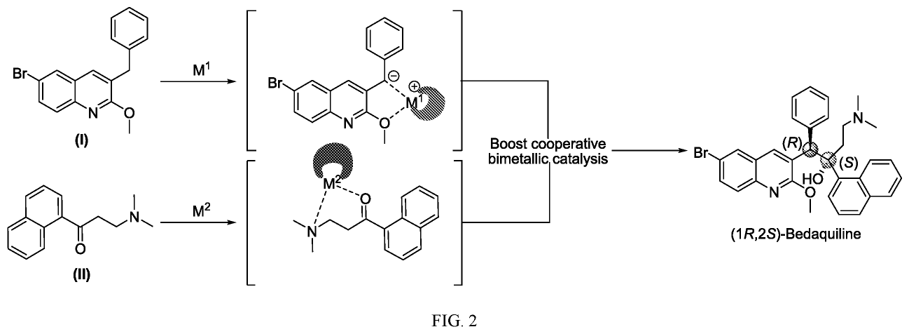

In the present invention, a method for asymmetric synthesis of (1R,2S)-bedaquiline using a chiral bimetallic cooperative catalysis system (FIG. 2) can be expressed by a reaction formula below:

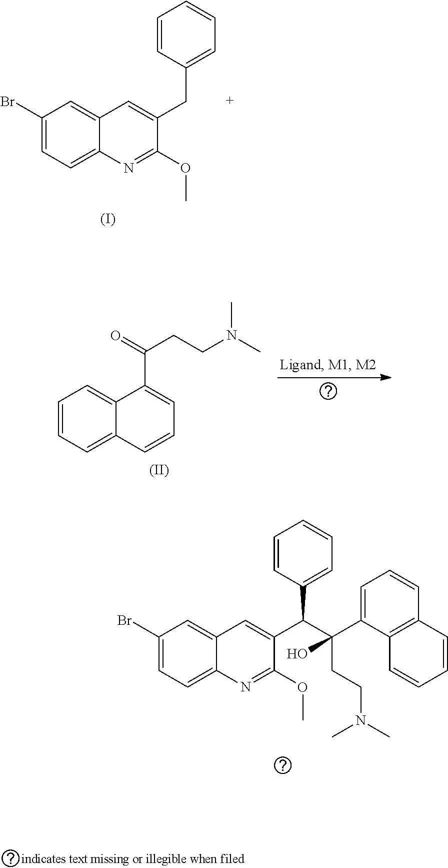

In the above reaction formula, “Ligand” refers to a ligand for activating metal M1 and M2 ions. The ligand has a structural formula of

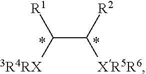

preferably, the chiral ligand may be selected from any one of the following formulas: L1-L12; and particularly, when the L1 is selected to carry out a reaction after detailed screening, optimal results can be obtained.

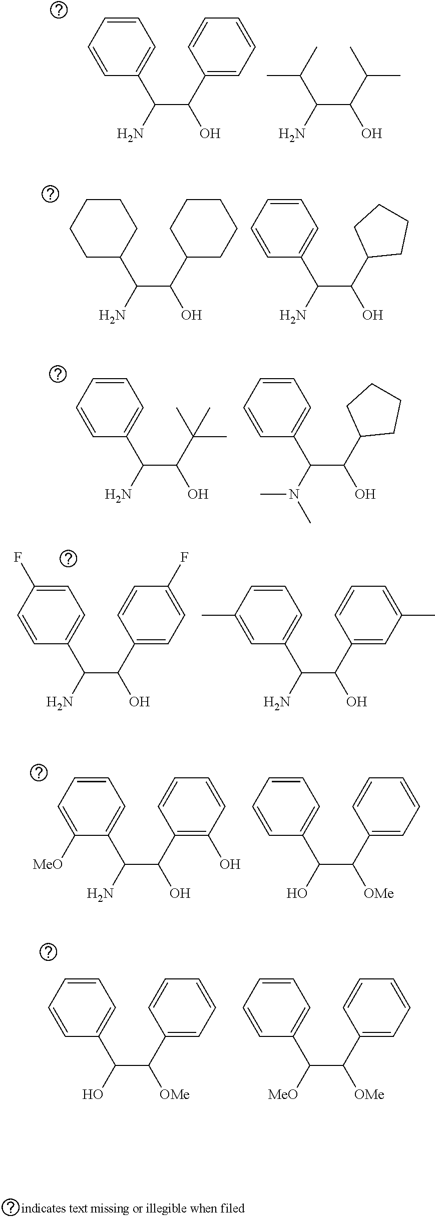

In the above reaction formula, “M1” refers to lithium, sodium or potassium. In a method for preparing bedaquiline based on chiral bimetallic cooperative catalysis in the present invention, the M1 is derived from one or a combination of n-butyllithium, sec-butyllithium, tert-butyllithium, lithium diisopropylamide, lithium tetramethylpiperidine, lithium bis(trimethylsilyl)amide, sodium bis(trimethylsilyl)amide, potassium bis(trimethylsilyl)amide and the like. The use amount of the M1 is 1-5 times of the equivalent of 6-bromo-3-benzyl-2-methoxyquinoline (I).

In the above reaction formula. “M2” refers to another metal cooperating with the M1 in the chiral bimetallic cooperative catalysis system of the present invention. The M2 is derived from MgCl₂, ZnCl₂, CuCl₂, AlCl₃, NiCl₂, CoCl₂, FeCl₃, NaCl, KCl, TiCl₄, CaCl₂, LiCl, LiBr. Lil, LiF and the like. The use amount of the M2 is 0.01-2.0 times of the equivalent of 3-dimethylamino-1-naphthyl-1-propanone (II).

In the above reaction formula, “Additive” refers to an additive in the chiral bimetallic cooperative catalysis system. In the method for preparing bedaquiline based on chiral bimetallic cooperative catalysis in the present invention, the additive is preferably selected from one of 1,8-diazabicyclo[5,4,0]undec-7-ene (DBU), 1,5-diazobicyclo[4.3.0]non-5-ene (DBN). N,N-diisopropylamine (DIPA), triethylenediamine (DABCO), 2,2,6,6-tetramethylpiperidine (TMP), hexamethylphosphoric triamide (HMPA), N,N,N′,N′-tetramethylethylenediamine (TMEDA) and 4-dimethylaminopyridine (DMAP). The use amount of the additive is 0.1-20 times of the equivalent of the 6-bromo-3-benzyl-2-methoxyquinoline (1).

In the above reaction formula, “Solvent” refers to a solvent capable of forming an organic solution of a system. In the method for preparing bedaquiline based on chiral bimetallic cooperative catalysis in the present invention, the solvent is not particularly limited as long as the solvent can promote the occurrence of a reaction to obtain a target product. However, based on the viewpoints of the reaction yield and the enantioselectivity and diastereoselectivity of a reaction, the solvent is preferably one or a mixture of more selected from ether solvents, such as ethyl ether, tetrahydrofuran, methyl tert-butyl ether, 1,4-dioxane, diisopropyl ether and ethylene glycol dimethyl ether, as well as toluene, xylene, n-hexane, cyclohexane, n-pentane and the like. Optimal results can be obtained by using tetrahydrofuran as a reaction solvent after screening.

In the following embodiments, the reaction temperature may be preferably a low temperature of −78° C. to 0° C., and the total reaction time may be set as 12 min to 26 h. Step 1:1-120 min is required for obtaining a chiral bimetallic cooperative catalysis system. Step 2:6-bromo-3-benzyl-2-methoxyquinoline (I) as a raw material is added to carry out a reaction for 10 min to 12 h. Step 3: An organic solution of 3-N,N-dimethylamino-1-naphthyl-1-propanone (II) is added to carry out another reaction for 10 min to 12 h.

In the following embodiments, the enantiomeric excess percentage (namely, ee value) is determined by high performance liquid chromatography (HPLC, chiral column). LC-2010 purchased from Shimadzo is used as an instrument for HPLC analysis, and specific operating conditions include: using a Chiralpak AD-H chiral chromatographic column produced by Daicel of Japan.

### Example 1

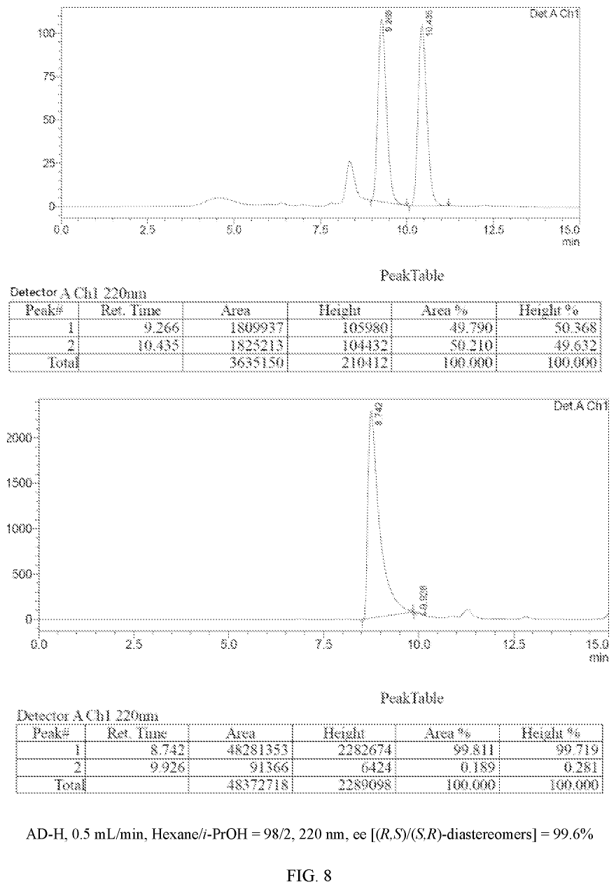

Under the protection of nitrogen, 67.8 mg (1.6 mmol, 2.0 equiv) of lithium chloride, 246.4 mg (1.2 mmol, 1.5 equiv) of (1S,2R)-2-amino-1,2-diphenylethanol (L1), 120 μL (0.8 mmol, 1.0 equiv) of DBU and 6 mL of anhydrous tetrahydrofuran were added into a 50 ml, dry Schlenk tube. A reaction flask was placed in a cold trap (−40° C.), 0.88 ml. (1.4 mmol) of an n-hexane solution of 1.6 M n-butyllithium was slowly added to carry out a reaction for 10 min, 0.77 mL (1.4 mmol) of a tetrahydrofuran/n-heptane/ethylbenzene solution of 2.0 M LDA was added dropwise to carry out another reaction at a temperature of −40° C. for half an hour, and then, 8 mL of a tetrahydrofuran (THF) solution of 262.6 mg (0.8 mmol, 1.0 equiv) of 3-benzyl-6-bromo-2-methoxyquinoline was slowly and completely added dropwise within 1 h. After the addition was completed, continuous stirring was performed for 2 h. Then, the temperature of the cold trap was lowered to ˜60° C., and a solution formed by 212.8 mg (0.96 mmol, 1.2 equiv) of 3-N,N-dimethylamino-1-naphtbyl-1-propanone and 6 mL of THE was slowly added within next 60 min, wherein the temperature of a reaction system was maintained at −59° C. to −62° C. in the addition process. After the addition was completed, a continuous reaction was carried out for 2 h. After the reaction was completed, a reaction solution was quenched with a saturated ammonium chloride solution at low temperature, transferred to a place at room temperature and extracted with ethyl acetate (3×5 mL), followed by column chromatography to obtain a pure product with a yield of 26%. According to determination of a crude product by nuclear magnetic resonance, the product has a dr value of 10:1. According to determination by HPLC, the product has an ee value of 99% (liquid chromatography spectra are as shown in FIG. 8).

### Example 2

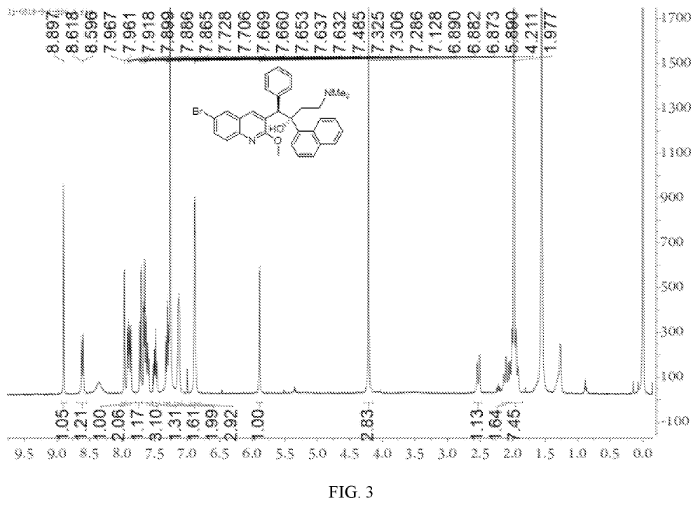

Under the protection of nitrogen, 138.9 mg (1.6 mmol, 2.0 equiv) of lithium bromide, 298.6 mg (1.4 mmol. 1.76 equiv) of (1S,2R)-2-amino-1,2-diphenylethanol (L1), 598 μL. (4.0 mmol, 5.0 equiv) of DBU and 6 mL of anhydrous tetrahydrofuran were added into a 50 ml, dry Schlenk tube. A reaction flask was placed in a cold trap (−20° C.). 0.88 mL (1.4 mmol) of an n-hexane solution of 1.6 M n-butyllithium was slowly added to carry out a reaction for 10 min, 0.77 ml (1.4 mmol) of a tetrahydrofuran/n-heptane/ethylbenzene solution of 2.0 M LDA was added dropwise to carry out another reaction at a temperature of −20° C. for 1 h, and then, 8 mL of a THF solution of 262.6 mg (0.8 mmol, 1.0 equiv) of 3-benzyl-6-bromo-2-methoxyquinoline was slowly and completely added dropwise within 2 h. After the addition was completed, continuous stirring was performed for 2 h. Then, the temperature of the cold trap was lowered to −78° C., and a solution formed by 212.8 mg (0.96 mmol, 1.2 equiv) of 3-N,N-dimethylamino-1-naphthyl-1-propanone and 6 ml of THE was slowly added within next 120 min, wherein the temperature of a reaction system was maintained at −78° C. in the addition process. After the addition was completed, a continuous reaction was carried out for 2 h. After the reaction was completed, a reaction solution was quenched with a saturated ammonium chloride solution at low temperature, transferred to a place at room temperature and extracted with ethyl acetate (3×5 mL), followed by column chromatography to obtain a pure product with a yield of 22%. According to determination of a crude product by nuclear magnetic resonance, the product has a dr value of 8:1. According to determination by HPLC, the product has an ee value of 99% (hydrogen nuclear magnetic resonance spectrum is as shown in FIG. 3).

### Example 3

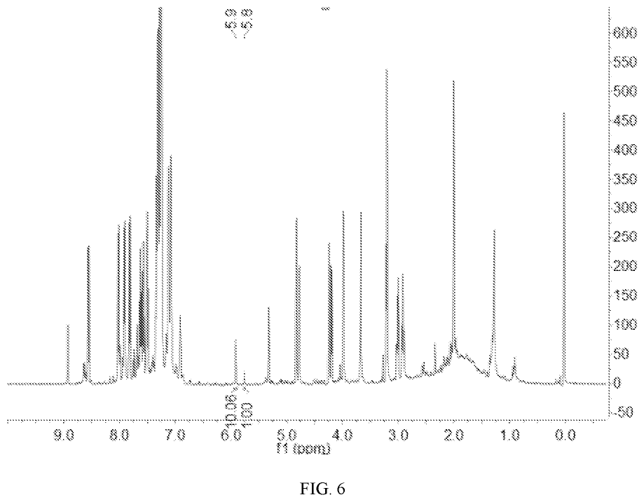

Under the protection of nitrogen, 81.4 mg (1.9 mmol, 2.4 equiv) of lithium chloride, 298.6 mg (1.4 mmol, 1.76 equiv) of (1S,2R)-2-amino-1,2-diphenylethanol (L1), 139 μl (0.8 mmol, 1.0 equiv) of HMPA and 6 mL of anhydrous tetrahydrofuran were added into a 50 mL dry Schlenk tube. A reaction flask was placed in a cold trap (−20° C.), 0.88 mL (1.4 mmol) of an n-hexane solution of 1.6 M n-butyllithium was slowly added to carry out a reaction for 10 min, 0.77 mL (1.4 mmol) of a tetrahydrofuran/n-heptane/ethylbenzene solution of 2.0 M LDA was added dropwise to carry out another reaction at a temperature of −20° C. for half an hour, and then, 8 ml of a THE solution of 262.6 mg (0.8 mmol, 1.0 equiv) of 3-benzyl-6-bromo-2-methoxyquinoline was slowly and completely added dropwise within 1 h. After the addition was completed, continuous stirring was performed for 2 h. Then, the temperature of the cold trap was lowered to ˜60° C., and a solution formed by 212.8 mg (0.96 mmol. 1.2 equiv) of 3-N,N-dimethylamino-1-naphthyl-1-propanone and 6 mL of THE was slowly added within next 90 min, wherein the temperature of a reaction system was maintained at −59° C. to −62° C. in the addition process. After the addition was completed, a continuous reaction was carried out for 2 h. After the reaction was completed, a reaction solution was quenched with a saturated ammonium chloride solution at low temperature, transferred to a place at room temperature and extracted with ethyl acetate (3×5 mL), followed by column chromatography to obtain a pure product with a yield of 22%. According to determination of a crude product by nuclear magnetic resonance, the product has a dr value of 10:1. According to determination by HPLC, the product has an ee value of 99% (hydrogen nuclear magnetic resonance spectrum is as shown in FIG. 6).

### Example 4

Under the protection of nitrogen, 81.4 mg (1.9 mmol, 2.4 equiv) of lithium chloride. 298.6 mg (1.4 mmol, 1.76 equiv) of (1S,2R)-2-amino-1,2-diphenylethanol (L1), 139 μl (0.8 mmol, 1.0 equiv) of HMPA and 6 ml, of anhydrous tetrahydrofuran were added into a 50 ml, dry Schlenk tube. A reaction flask was placed in a cold trap (−20° C.), 0.88 mL. (1.4 mmol) of an n-hexane solution of 1.6 M n-butyllithium was slowly added to carry out a reaction for 1 min, 0.77 mL (1.4 mmol) of a tetrahydrofuran/n-heptane/ethylbenzene solution of 2.0 M LDA was added dropwise to carry out another reaction at a temperature of −20° C. for half an hour, and then, 8 mL, of a THE solution of 262.6 mg (0.8 mmol, 1.0 equiv) of 3-benzyl-6-bromo-2-methoxyquinoline was slowly and completely added dropwise within 1 h. After the addition was completed, continuous stirring was performed for 2 h. Then, the temperature of the cold trap was lowered to −60° C., and a solution formed by 212.8 mg (0.96 mmol, 1.2 equiv) of 3-N,N-dimethylamino-1-naphthyl-1-propanone and 6 mL of THE was slowly added within next 90 min, wherein the temperature of a reaction system was maintained at −59° C. to −62° C.′ in the addition process. After the addition was completed, a continuous reaction was carried out for 3 h. After the reaction was completed, a reaction solution was quenched with a saturated ammonium chloride solution at low temperature, transferred to a place at room temperature and extracted with ethyl acetate (3×5 mL), followed by column chromatography to obtain a pure product with a yield of 18%. According to determination of a crude product by nuclear magnetic resonance, the product has a dr value of 8:1. According to determination by HPLC, the product has an ee value of 98%.

### Example 5

Under the protection of nitrogen. 81.4 mg (1.9 mmol, 2.4 equiv) of lithium chloride, 298.6 mg (1.4 mmol, 1.76 equiv) of (1S,2R)-2-amino-1,2-diphenylethanol (L1), 139 μL (0.8 mmol, 1.0 equiv) of HMPA and 6 ml of anhydrous tetrahydrofuran were added into a 50 mL dry Schlenk tube. A reaction flask was placed in a cold trap (−20° C.), 0.88 mL. (1.4 mmol) of an n-hexane solution of 1.6 M n-butyllithium was slowly added to carry out a reaction for 1 min, 0.77 ml. (1.4 mmol) of a tetrahydrofuran/n-heptane/ethylbenzene solution of 2.0 M LDA was added dropwise to carry out another reaction at a temperature of −20° C. for half an hour, and then, 8 ml of a THE solution of 262.6 mg (0.8 mmol, 1.0 equiv) of 3-benzyl-6-bromo-2-methoxyquinoline was slowly and completely added dropwise within 1 h. After the addition was completed, continuous stirring was performed for 10 min. Then, the temperature of the cold trap was lowered to −60° C., and a solution formed by 212.8 mg (0.96 mmol, 1.2 equiv) of 3-N,N-dimethylamino-1-naphthyl-1-propanone and 6 ml of THE was slowly added within next 90 min, wherein the temperature of a reaction system was maintained at −59° C. to −62° C. in the addition process. After the addition was completed, a continuous reaction was carried out for 12 h. After the reaction was completed, a reaction solution was quenched with a saturated ammonium chloride solution at low temperature, transferred to a place at room temperature and extracted with ethyl acetate (3×5 mL), followed by column chromatography to obtain a pure product with a yield of 22%. According to determination of a crude product by nuclear magnetic resonance, the product has a dr value of 6:1. According to determination by HPLC, the product has an ee value of 98%.

### Example 6

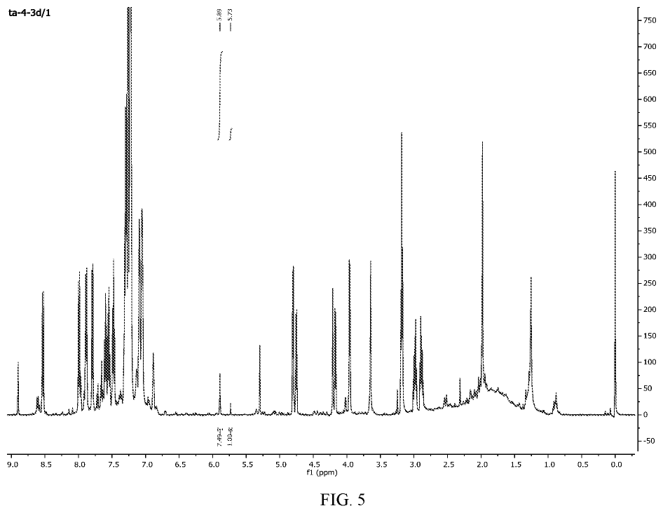

Under the protection of nitrogen, 81.4 mg (1.9 mmol, 2.4 equiv) of lithium chloride, 298.6 mg (1.4 mmol, 1.76 equiv) of (1S,2R)-2-amino-1,2-diphenylethanol (L1), 139 μL (0.8 mmol, 1.0 equiv) of HMPA and 6 mL of anhydrous tetrahydrofuran were added into a 50 mL dry Schlenk tube. A reaction flask was placed in a cold trap (−20° C.). 0.88 ml (1.4 mmol) of an n-hexane solution of 1.6 M n-butyllithium was slowly added to carry out a reaction for 5 min, 0.77 mL. (1.4 mmol) of a tetrahydrofuran/n-heptane/ethylbenzene solution of 2.0 M LDA was added dropwise to carry out another reaction at a temperature of −20° C. for half an hour, and then, 8 mL of a THE solution of 262.6 mg (0.8 mmol, 1.0 equiv) of 3-benzyl-6-bromo-2-methoxyquinoline was slowly and completely added dropwise within 1 h. After the addition was completed, continuous stirring was performed for 12 h. Then, the temperature of the cold trap was lowered to −60° C., and a solution formed by 212.8 mg (0.96 mmol, 1.2 equiv) of 3-N,N-dimethylamino-1-naphthyl-1-propanone and 6 mL of THE was slowly added within next 90 min, wherein the temperature of a reaction system was maintained at −59° C. to −62° C. in the addition process. After the addition was completed, a continuous reaction was carried out for 12 h. After the reaction was completed, a reaction solution was quenched with a saturated ammonium chloride solution at low temperature, transferred to a place at room temperature and extracted with ethyl acetate (3×5 mL), followed by column chromatography to obtain a pure product with a yield of 29%. According to determination of a crude product by nuclear magnetic resonance, the product has a dr value of 7:1. According to determination by HPLC, the product has an ee value of 99% (hydrogen nuclear magnetic resonance spectrum is as shown in FIG. 5).

### Example 7

Under the protection of nitrogen, 81.4 mg (1.9 mmol, 2.4 equiv) of lithium chloride, 298.6 mg (1.4 mmol, 1.76 equiv) of (1S,2R)-2-amino-1,2-diphenylethanol (L1), 139 μL (0.8 mmol, 1.0 equiv) of HMPA and 6 mL of anhydrous tetrahydrofuran were added into a 50 mL dry Schlenk tube. A reaction flask was placed in a cold trap (−20° C.), 0.88 ml. (1.4 mmol) of an n-hexane solution of 1.6 M n-butyllithium was slowly added to carry out a reaction for 5 min, 0.77 ml. (1.4 mmol) of a tetrahydrofuran/n-heptane/ethylbenzene solution of 2.0 M LDA was added dropwise to carry out another reaction at a temperature of −20° C. for half an hour, and then, 8 mL of a THE solution of 262.6 mg (0.8 mmol, 1.0 equiv) of 3-benzyl-6-bromo-2-methoxyquinoline was slowly and completely added dropwise within 1 h. After the addition was completed, continuous stirring was performed for 10 min. Then, the temperature of the cold trap was lowered to −60° C., and a solution formed by 212.8 mg (0.96 mmol, 1.2 equiv) of 3-N,N-dimethylamino-1-naphthyl-1-propanone and 6 ml of THE was slowly added within next 90 min, wherein the temperature of a reaction system was maintained at −59° C. to −62° C. in the addition process. After the addition was completed, a continuous reaction was carried out for 12 h. After the reaction was completed, a reaction solution was quenched with a saturated ammonium chloride solution at low temperature, transferred to a place at room temperature and extracted with ethyl acetate (3×5 mL), followed by column chromatography to obtain a pure product with a yield of 19%. According to determination of a crude product by nuclear magnetic resonance, the product has a dr value of 7:1. According to determination by HPLC, the product has an ee value of 97%.

### Example 8

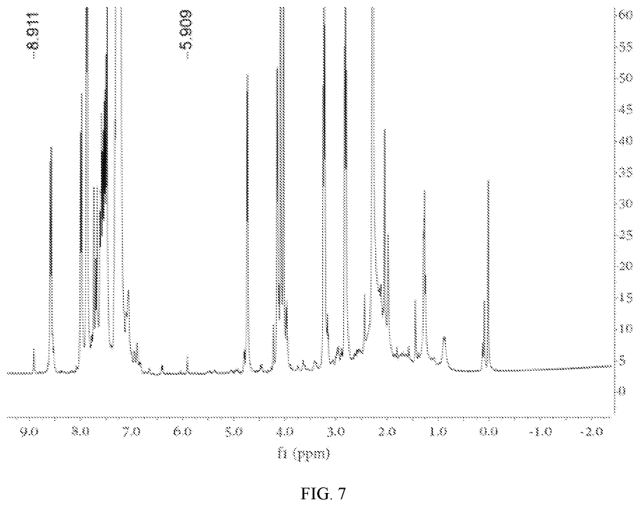

Under the protection of nitrogen, 81.4 mg (1.9 mmol, 2.4 equiv) of lithium chloride, 298.6 mg (1.4 mmol, 1.76 equiv) of (1S,2R)-2-amino-1,2-diphenylethanol (L1), 696 μL (4.0 mmol, 5.0 equiv) of HMPA and 6 mL of anhydrous tetrahydrofuran were added into a 50 mL dry Schlenk tube. A reaction flask was placed in a cold trap (−30° C.), 0.88 mL (1.4 mmol) of an n-hexane solution of 1.6 M n-butyllithium was slowly added to carry out a reaction for 10 min, 0.77 ml. (1.4 mmol) of a tetrahydrofuran/n-heptane/ethylbenzene solution of 2.0 M LDA was added dropwise to carry out another reaction at a temperature of −30° C. for 1 h, and then, 8 ml of a THE solution of 262.6 mg (0.8 mmol, 1.0 equiv) of 3-benzyl-6-bromo-2-methoxyquinoline was slowly and completely added dropwise within 1 h. After the addition was completed, continuous stirring was performed for 2 h. Then, the temperature of the cold trap was lowered to −50° C., and a solution formed by 212.8 mg (0.96 mmol, 1.2 equiv) of 3-N,N-dimethylamino-1-naphtbyl-1-propanone and 6 mL of THE was slowly added within next 60 min, wherein the temperature of a reaction system was maintained at −50° C. in the addition process. After the addition was completed, a continuous reaction was carried out for 2 h. After the reaction was completed, a reaction solution was quenched with a saturated ammonium chloride solution at low temperature, transferred to a place at room temperature and extracted with ethyl acetate (3×5 mL), followed by column chromatography to obtain a pure product with a yield of 18%. According to determination of a crude product by nuclear magnetic resonance, the product has a dr value of 20:1. According to determination by HPLC, the product has an ee value of 99% (hydrogen nuclear magnetic resonance spectrum is as shown in FIG. 7).

### Example 9

Under the protection of nitrogen, 81.4 mg (1.9 mmol, 2.4 equiv) of lithium chloride, 298.6 mg (1.4 mmol, 1.76 equiv) of (1S,2R)-2-amino-1,2-diphenylethanol (L1), 120 μL (0.8 mmol, 1.0 equiv) of DBU and 6 mL of anhydrous tetrahydrofuran were added into a 50 mL dry Schlenk tube. A reaction flask was placed in a cold trap (−30° C.), 0.88 mL (1.4 mmol) of an n-hexane solution of 1.6 M n-butyllithium was slowly added to carry out a reaction for 10 min, 0.77 ml. (1.4 mmol) of a tetrahydrofuran/n-heptane/ethylbenzene solution of 2.0 M LDA was added dropwise to carry out another reaction at a temperature of −30° C. for half an hour, and then, 8 ml of a THE solution of 262.6 mg (0.8 mmol, 1.0 equiv) of 3-benzyl-6-bromo-2-methoxyquinoline was slowly and completely added dropwise within 1 h. After the addition was completed, continuous stirring was performed for 2 h. Then, the temperature of the cold trap was lowered to −60° C., and a solution formed by 212.8 mg (0.96 mmol, 1.2 equiv) of 3-N,N-dimethylamino-1-naphthyl-1-propanone and 6 mL of THE was slowly added within next 60 min, wherein the temperature of a reaction system was maintained at −60° C. in the addition process. After the addition was completed, a continuous reaction was carried out for 2 h. After the reaction was completed, a reaction solution was quenched with a saturated ammonium chloride solution at low temperature, transferred to a place at room temperature and extracted with ethyl acetate (3×5 mL), followed by column chromatography to obtain a pure product with a yield of 25%. According to determination of a crude product by nuclear magnetic resonance, the product has a dr value of 13:1. According to determination by HPLC, the product has an ee value of 99%.

### Example 10

Under the protection of nitrogen, 152 mg (1.6 mmol, 2.0 equiv) of magnesium chloride, 298.6 mg (1.4 mmol, 1.76 equiv) of (1S,2R)-2-amino-1,2-diphenylethanol (L1), 120 μL (0.8 mmol, 1.0 equiv) of TMEDA and 6 mL of anhydrous tetrahydrofuran were added into a 50 mL dry Schlenk tube. A reaction flask was placed in a cold trap (−30° C.), 0.88 mL (1.4 mmol) of an n-hexane solution of 1.6 M n-butyllithium was slowly added to carry out a reaction for 10 min, 0.77 ml (1.4 mmol) of a tetrahydrofuran/n-heptane/ethylbenzene solution of 2.0 M LDA was added dropwise to carry out another reaction at a temperature of −30° C. for half an hour, and then, 8 mL of a THE solution of 262.6 mg (0.8 mmol, 1.0 equiv) of 3-benzyl-6-bromo-2-methoxyquinoline was slowly and completely added dropwise within 1 h. After the addition was completed, continuous stirring was performed for 2 h. Then, the temperature of the cold trap was lowered to −60° C., and a solution formed by 212.8 mg (0.96 mmol, 1.2 equiv) of 3-N,N-dimethylamino-1-naphthyl-1-propanone and 6 mL of THE was slowly added within next 60 min, wherein the temperature of a reaction system was maintained at −80° C.′ in the addition process. After the addition was completed, a continuous reaction was carried out for 2 h. After the reaction was completed, a reaction solution was quenched with a saturated ammonium chloride solution at low temperature, transferred to a place at room temperature and extracted with ethyl acetate (3*5 mL), followed by column chromatography to obtain a pure product with a yield of 34%. According to determination of a crude product by nuclear magnetic resonance, the product has a dr value of 2:1. According to determination by HPLC, the product has an ee value of 95%.

### Example 11

Under the protection of nitrogen, 81.4 mg (1.9 mmol, 2.4 equiv) of lithium chloride, 492.7 mg (2.4 mmol, 3.0 equiv) of (1S,2R)-2-amino-1,2-diphenylethanol (L1), 240 μl. (1.6 mmol, 2.0 equiv) of TMEDA and 6 ml of anhydrous tetrahydrofuran were added into a 50 mL dry Schlenk tube. A reaction flask was placed in a cold trap (−50° C.), 0.88 ml. (1.4 mmol) of an n-hexane solution of 1.6 M n-butyllithium was slowly added to carry out a reaction for 10 min, 0.77 ml (1.4 mmol) of a tetrahydrofuran/n-heptane/ethylbenzene solution of 2.0 M LDA was added dropwise to carry out another reaction at a temperature of −50° C. for half an hour, and then, 8 mL of a THE solution of 262.6 mg (0.8 mmol. 1.0 equiv) of 3-benzyl-6-bromo-2-methoxyquinoline was slowly and completely added dropwise within 1 h. After the addition was completed, continuous stirring was performed for 2 h. Then, the temperature of the cold trap was lowered to −60° C., and a solution formed by 212.8 mg (0.96 mmol, 1.2 equiv) of 3-N,N-dimethylamino-1-naphthyl-1-propanone and 6 mL of THE was slowly added within next 60 min, wherein the temperature of a reaction system was maintained at −50° C. in the addition process. After the addition was completed, a continuous reaction was carried out for 2 b. After the reaction was completed, a reaction solution was quenched with a saturated ammonium chloride solution at low temperature, transferred to a place at room temperature and extracted with ethyl acetate (3×5 mL), followed by column chromatography to obtain a pure product with a yield of 29%. According to determination of a crude product by nuclear magnetic resonance, the product has a dr value of 3:1. According to determination by HPLC, the product has an ee value of 97%.

### Example 12

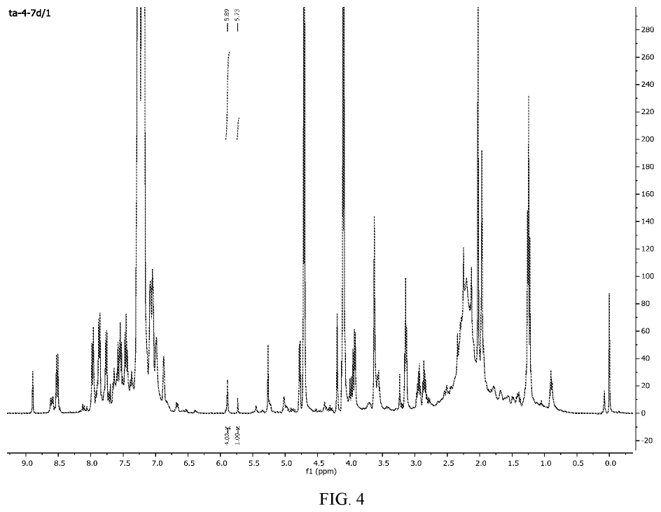

Under the protection of nitrogen, 81.4 mg (1.9 mmol, 2.4 equiv) of lithium chloride, 298.6 mg (1.4 mmol, 1.76 equiv) of (1S,2R)-2-amino-1,2-diphenylethanol (L1). 600 μL (4.0 mmol, 5.0 equiv) of TMEDA and 6 mL of anhydrous tetrahydrofuran were added into a 50 mL dry Schlenk tube. A reaction flask was placed in a cold trap (0° C.), 0.88 ml. (1.4 mmol) of an n-hexane solution of 1.6 M n-butyllithium was slowly added to carry out a reaction for 10 min, 0.77 mL (1.4 mmol) of a tetrahydrofuran/n-heptane/ethylbenzene solution of 2.0 M LDA was added dropwise to carry out another reaction at a temperature of −20° C. for half an hour, and then, 8 ml of a THE solution of 262.6 mg (0.8 mmol, 1.0 equiv) of 3-benzyl-6-bromo-2-methoxyquinoline was slowly and completely added dropwise within 1 h. After the addition was completed, continuous stirring was performed for 2 h. Then, the temperature of the cold trap was lowered to −60° C., and a solution formed by 212.8 mg (0.96 mmol. 1.2 equiv) of 3-N,N-dimethylamino-1-naphthyl-1-propanone and 6 mL of THE was slowly added within next 60 min, wherein the temperature of a reaction system was maintained at −78° C. in the addition process. After the addition was completed, a continuous reaction was carried out for 2 h. After the reaction was completed, a reaction solution was quenched with a saturated ammonium chloride solution at low temperature, transferred to a place at room temperature and extracted with ethyl acetate (3×5 mL), followed by column chromatography to obtain a pure product with a yield of 28%. According to determination of a crude product by nuclear magnetic resonance, the product has a dr value of 4:1. According to determination by HPLC, the product has an ee value of 98% (hydrogen nuclear magnetic resonance spectrum is as shown in FIG. 4).

### Example 13

Under the protection of nitrogen, 81.4 mg (1.9 mmol, 2.4 equiv) of lithium chloride, 298.6 mg (1.4 mmol, 1.76 equiv) of (1S,2R)-2-amino-1,2-diphenylethanol (L1), 0.88 μL (8.0 mmol, 10.0 equiv) of DABCO and 6 mL of anhydrous tetrahydrofuran were added into a 50 ml dry Schlenk tube. A reaction flask was placed in a cold trap (−10° C.), 0.88 ml. (1.4 mmol) of an n-hexane solution of 1.6 M n-butyllithium was slowly added to carry out a reaction for 10 min, 0.77 mL (1.4 mmol) of a tetrahydrofuran/n-heptane/ethylbenzene solution of 2.0 M LDA was added dropwise to carry out another reaction at a temperature of −10° C. for half an hour, and then, 8 ml of a THE solution of 262.6 mg (0.8 mmol, 1.0 equiv) of 3-benzyl-6-bromo-2-methoxyquinoline was slowly and completely added dropwise within 1 h. After the addition was completed, continuous stirring was performed for 2 h. Then, the temperature of the cold trap was lowered to −60° C., and a solution formed by 212.8 mg (0.96 mmol, 1.2 equiv) of 3-N,N-dimethylamino-1-naphthyl-1-propanone and 6 mL of THE was slowly added within next 60 min, wherein the temperature of a reaction system was maintained at −78° C. in the addition process. After the addition was completed, a continuous reaction was carried out for 2 h. After the reaction was completed, a reaction solution was quenched with a saturated ammonium chloride solution at low temperature, transferred to a place at room temperature and extracted with ethyl acetate (3×5 mL), followed by column chromatography to obtain a pure product with a yield of 25%. According to determination of a crude product by nuclear magnetic resonance, the product has a dr value of 2:1. According to determination by HPLC, the product has an ee value of 92%.

### Example 14

Under the protection of nitrogen, 59.1 mg (0.8 mmol, 1.0 equiv) of sodium chloride. 298.6 mg (1.4 mmol, 1.76 equiv) of (1S,2R)-2-amino-1,2-diphenylethanol (L1), 176 μL (1.6 mmol, 2.0 equiv) of DABCO and 6 mL of anhydrous tetrahydrofuran were added into a 50 mL dry Schlenk tube. A reaction flask was placed in a cold trap (−20° C.), 0.88 mL (1.4 mmol) of an n-hexane solution of 1.6 M n-butyllithium was slowly added to carry out a reaction for 10 min, 0.77 mL (1.4 mmol) of a tetrahydrofuran/n-heptane/ethylbenzene solution of 2.0 M LDA was added dropwise to carry out another reaction at a temperature of −20° C. for half an hour, and then, 8 ml, of a THE solution of 262.6 mg (0.8 mmol, 1.0 equiv) of 3-benzyl-6-bromo-2-methoxyquinoline was slowly and completely added dropwise within 1 b. After the addition was completed, continuous stirring was performed for 2 h. Then, the temperature of the cold trap was lowered to ˜60° C., and a solution formed by 212.8 mg (0.96 mmol, 1.2 equiv) of 3-N,N-dimethylamino-1-naphthyl-1-propanone and 6 mL of THE was slowly added within next 60 min, wherein the temperature of a reaction system was maintained at −50° C. in the addition process. After the addition was completed, a continuous reaction was carried out for 2 h. After the reaction was completed, a reaction solution was quenched with a saturated ammonium chloride solution at low temperature, transferred to a place at room temperature and extracted with ethyl acetate (3×5 ml), followed by column chromatography to obtain a pure product with a yield of 22%. According to determination of a crude product by nuclear magnetic resonance, the product has a dr value of 2:1. According to determination by HPLC, the product has an ee value of 95%.

### Example 15

Under the protection of nitrogen. 81.4 mg (1.9 mmol, 2.4 equiv) of lithium chloride, 298.6 mg (1.4 mmol, 1.76 equiv) of (1S,2R)-2-amino-1,2-diphenylethanol (L1), 440 μl (4.0 mmol, 5.0 equiv) of DABCO and 6 mL, of anhydrous tetrahydrofuran were added into a 50 mL dry Schlenk tube. A reaction flask was placed in a cold trap (−30° C.), 0.88 mL (1.4 mmol) of an n-hexane solution of 1.6 M n-butyllithium was slowly added to carry out a reaction for 10 min, 0.77 mL. (1.4 mmol) of a tetrahydrofuran/n-heptane/ethylbenzene solution of 2.0 M LDA was added dropwise to carry out another reaction at a temperature of −30° C. for half an hour, and then, 8 mL of a THE solution of 262.6 mg (0.8 mmol, 1.0 equiv) of 3-benzyl-6-bromo-2-methoxyquinoline was slowly and completely added dropwise within 1 b. After the addition was completed, continuous stirring was performed for 2 h. Then, the temperature of the cold trap was lowered to −60° C., and a solution formed by 212.8 mg (0.96 mmol, 1.2 equiv) of 3-N,N-dimethylamino-1-naphthyl-1-propanone and 6 mL of THE was slowly added within next 60 min, wherein the temperature of a reaction system was maintained at −78° C. in the addition process. After the addition was completed, a continuous reaction was carried out for 2 h. After the reaction was completed, a reaction solution was quenched with a saturated ammonium chloride solution at low temperature, transferred to a place at room temperature and extracted with ethyl acetate (3×5 mL), followed by column chromatography to obtain a pure product with a yield of 25%. According to determination of a crude product by nuclear magnetic resonance, the product has a dr value of 4:1. According to determination by HPLC, the product has an ee value of 92%.

### Example 16

Under the protection of nitrogen, 81.4 mg (1.9 mmol, 2.4 equiv) of lithium chloride, 298.6 mg (1.4 mmol, 1.76 equiv) of (1S,2R)-2-amino-1,2-diphenylethanol (L1), 98 mg (0.8 mmol. 1.0 equiv) of DMAP and 6 mL of anhydrous tetrahydrofuran were added into a 50 ml, dry Schlenk tube. A reaction flask was placed in a cold trap (−20° C.), 0.88 mL. (1.4 mmol) of an n-hexane solution of 1.6 M n-butyllithium was slowly added to carry out a reaction for 10 min, 0.77 mL (1.4 mmol) of a tetrahydrofuran/n-heptane/ethylbenzene solution of 2.0 M LDA was added dropwise to carry out another reaction at a temperature of −20° C. for half an hour, and then, 8 mL of a THE solution of 262.6 mg (0.8 mmol, 1.0 equiv) of 3-benzyl-6-bromo-2-methoxyquinoline was slowly and completely added dropwise within 1 h. After the addition was completed, continuous stirring was performed for 2 h. Then, the temperature of the cold trap was lowered to −60° C., and a solution formed by 212.8 mg (0.96 mmol, 1.2 equiv) of 3-N,N-dimethylamino-1-naphthyl-1-propanone and 6 ml of THF was slowly added within next 60 min, wherein the temperature of a reaction system was maintained at −80° C. in the addition process. After the addition was completed, a continuous reaction was carried out for 2 h. After the reaction was completed, a reaction solution was quenched with a saturated ammonium chloride solution at low temperature, transferred to a place at room temperature and extracted with ethyl acetate (3×5 ml), followed by column chromatography to obtain a pure product with a yield of 35%. According to determination of a crude product by nuclear magnetic resonance, the product has a dr value of 3:1. According to determination by HPLC, the product has an ee value of 94%.

### Example 17

Under the protection of nitrogen, 81.4 mg (1.9 mmol, 2.4 equiv) of lithium chloride, 232.4 mg (1.6 mmol, 2.0 equiv) of (3S,4R)-4-amino-2,5-dimethylhexanal (L2), 98 mg (0.8 mmol, 1.0 equiv) of DMAP and 6 mL of anhydrous tetrahydrofuran were added into a 50 mL dry Schlenk tube. A reaction flask, was placed in a cold trap (−10° C.), 0.88 mL. (1.4 mmol) of an n-hexane solution of 1.6 M n-butyllithium was slowly added to carry out a reaction for 10 min, 0.77 ml (1.4 mmol) of a tetrahydrofuran/n-heptane/ethylbenzene solution of 2.0 M LDA was added dropwise to carry out another reaction at a temperature of −10° C. for half an hour, and then, 8 ml of a THE solution of 262.6 mg (0.8 mmol, 1.0 equiv) of 3-benzyl-6-bromo-2-methoxyquinoline was slowly and completely added dropwise within 1 h. After the addition was completed, continuous stirring was performed for 1 h. Then, the temperature of the cold trap was lowered to −20° C., and a solution formed by 212.8 mg (0.96 mmol, 1.2 equiv) of 3-N,N-dimethylamino-1-naphthyl-1-propanone and 6 mL of THE was slowly added within next 60 min, wherein the temperature of a reaction system was maintained at −80° C. in the addition process. After the addition was completed, a continuous reaction was carried out for 4 h. After the reaction was completed, a reaction solution was quenched with a saturated ammonium chloride solution at low temperature, transferred to a place at room temperature and extracted with ethyl acetate (3×5 mL), followed by column chromatography to obtain a pure product with a yield of 37%. According to determination of a crude product by nuclear magnetic resonance, the product has a dr value of 4:1. According to determination by HPLC, the product has an ee value of 91%.

### Example 18

Under the protection of nitrogen, 81.4 mg (1.9 mmol, 2.4 equiv) of lithium chloride, 450.8 mg (2.0 mmol, 2.5 equiv) of (1 S,2R)-2-amino-1,2-dicyclohexylmethanol (13), 14 μL (0.08 mmol, 0.1 equiv) of HMPA and 6 mL of anhydrous tetrahydrofuran were added into a 50 ml dry Schlenk tube. A reaction flask was placed in a cold trap (−10° C.), 0.88 ml (1.4 mmol) of an n-hexane solution of 1.6 M n-butyllithium was slowly added to carry out a reaction for 10 min, 0.77 ml. (1.4 mmol) of a tetrahydrofuran/n-heptane/ethylbenzene solution of 2.0 M LDA was added dropwise to carry out another reaction at a temperature of −10° C. for half an hour, and then, 8 mL of a THE solution of 262.6 mg (0.8 mmol, 1.0 equiv) of 3-benzyl-6-bromo-2-methoxyquinoline was slowly and completely added dropwise within 1 h. After the addition was completed, continuous stirring was performed for 1 h. Then, the temperature of the cold trap was lowered to −20° C., and a solution formed by 212.8 mg (0.96 mmol, 1.2 equiv) of 3-N,N-dimethylamino-1-naphthyl-1-propanone and 6 mL of THE was slowly added within next 60 min, wherein the temperature of a reaction system was maintained at −80° C. in the addition process. After the addition was completed, a continuous reaction was carried out for 4 h. After the reaction was completed, a reaction solution was quenched with a saturated ammonium chloride solution at low temperature, transferred to a place at room temperature and extracted with ethyl acetate (3×5 ml), followed by column chromatography to obtain a pure product with a yield of 37%. According to determination of a crude product by nuclear magnetic resonance, the product has a dr value of 4:1. According to determination by HPLC, the product has an ee value of 53%.

### Example 19

Under the protection of nitrogen, 81.4 mg (1.9 mmol, 2.4 equiv) of lithium chloride, 82.1 mg (0.4 mmol, 0.5 equiv) of (1S,2R)-2-amino-1-Cyclopentyl-2-phenylethanol (14), 2.4 mL (16.0 mmol, 20.0 equiv) of TMEDA and 6 mL of anhydrous tetrahydrofuran were added into a 50 ml, dry Schlenk tube. A reaction flask was placed in a cold trap (−10° C.), 0.88 mL. (1.4 mmol) of an n-hexane solution of 1.6 M n-butyllithium was slowly added to carry out a reaction for 10 min, 0.77 ml. (1.4 mmol) of a tetrahydrofuran/n-heptane/ethylbenzene solution of 2.0 M LDA was added dropwise to carry out another reaction at a temperature of −10° C. for half an hour, and then, 8 ml, of a THE solution of 262.6 mg (0.8 mmol, 1.0 equiv) of 3-benzyl-6-bromo-2-methoxyquinoline was slowly and completely added dropwise within 1 h. After the addition was completed, continuous stirring was performed for 1 h. Then, the temperature of the cold trap was lowered to −20° C., and a solution formed by 212.8 mg (0.96 mmol, 1.2 equiv) of 3-N,N-dimethylamino-1-naphthyl-1-propanone and 6 mL, of THE was slowly added within next 60 min, wherein the temperature of a reaction system was maintained at −80° C. in the addition process. After the addition was completed, a continuous reaction was carried out for 4 h. After the reaction was completed, a reaction solution was quenched with a saturated ammonium chloride solution at low temperature, transferred to a place at room temperature and extracted with ethyl acetate (3×5 mL), followed by column chromatography to obtain a pure product with a yield of 37%. According to determination of a crude product by nuclear magnetic resonance, the product has a dr value of 1:1. According to determination by HPLC, the product has an ee value of 56%.

### Example 20

Under the protection of nitrogen, 81.4 mg (1.9 mmol, 2.4 equiv) of lithium chloride, 231.9 mg (1.2 mmol, 1.5 equiv) of (1R,2S)-1-amino-3,3-dimethyl-1-phenylbutanol (L5), 98 mg (0.8 mmol, 1.0 equiv) of DMAP and 6 mL of anhydrous tetrahydrofuran were added into a 50 mL dry Schlenk tube. A reaction flask was placed in a cold trap (−10° C.), 0.88 mL (1.4 mmol) of an n-hexane solution of 1.6 M n-butyllithium was slowly added to carry out a reaction for 10 min, 0.77 mL (1.4 mmol) of a tetrahydrofuran/n-heptane/ethylbenzene solution of 2.0 M LDA was added dropwise to carry out another reaction at a temperature of −10° C. for half an hour, and then, 8 ml, of a THE solution of 262.6 mg (0.8 mmol, 1.0 equiv) of 3-benzyl-6-bromo-2-methoxyquinoline was slowly and completely added dropwise within 1 h. After the addition was completed, continuous stirring was performed for 1 h. Then, the temperature of the cold trap was lowered to ˜20° C., and a solution formed by 212.8 mg (0.96 mmol, 1.2 equiv) of 3-N,N-dimethylamino-1-naphthyl-1-propanone and 6 ml of THE was slowly added within next 60 min, wherein the temperature of a reaction system was maintained at −80° C. in the addition process. After the addition was completed, a continuous reaction was carried out for 4 h. After the reaction was completed, a reaction solution was quenched with a saturated ammonium chloride solution at low temperature, transferred to a place at room temperature and extracted with ethyl acetate (3×5 mL), followed by column chromatography to obtain a pure product with a yield of 37%. According to determination of a crude product by nuclear magnetic resonance, the product has a dr value of 1:1. According to determination by HPLC, the product has an ee value of 27%.

### Example 21

Under the protection of nitrogen, 81.4 mg (1.9 mmol, 2.4 equiv) of lithium chloride, 149.3 mg (0.64 mmol, 0.8 equiv) of (1S,2R)-1-Cyclopentyl-2-dimethylamino-2-phenylethanol (L6), 98 mg (0.8 mmol, 1.0 equiv) of DMAP and 6 ml of anhydrous tetrahydrofuran were added into a 50 ml dry Schlenk tube. A reaction flask was placed in a cold trap (−10° C.), 0.88 mL (1.4 mmol) of an n-hexane solution of 1.6 M n-butyllithium was slowly added to carry out a reaction for 10 min, 0.77 ml (1.4 mmol) of a tetrahydrofuran/n-heptane/ethylbenzene solution of 2.0 M LDA was added dropwise to carry out another reaction at a temperature of −10° C. for half an hour, and then, 8 mL of a THE solution of 262.6 mg (0.8 mmol. 1.0 equiv) of 3-benzyl-6-bromo-2-methoxyquinoline was slowly and completely added dropwise within 1 h. After the addition was completed, continuous stirring was performed for 1 h. Then, the temperature of the cold trap was lowered to −20° C., and a solution formed by 212.8 mg (0.96 mmol. 1.2 equiv) of 3-N,N-dimethylamino-1-naphthyl-1-propanone and 6 mL of THF was slowly added within next 60 min, wherein the temperature of a reaction system was maintained at −80° C. in the addition process. After the addition was completed, a continuous reaction was carried out for 4 h. After the reaction was completed, a reaction solution was quenched with a saturated ammonium chloride solution at low temperature, transferred to a place at room temperature and extracted with ethyl acetate (3×5 ml), followed by column chromatography to obtain a pure product with a yield of 34%. According to determination of a crude product by nuclear magnetic resonance, the product has a dr value of 1.6:1. According to determination by HPLC, the product has an ee value of 49%.

### Example 22

Under the protection of nitrogen, 180.9 mg (1.9 mmol, 2.4 equiv) of magnesium chloride, 574.8 mg (2.8 mmol, 3.5 equiv) of (1S,2R)-2-amino-1,2-di-p-fluoro phenylethanol (L7), 98 mg (0.8 mmol, 1.0 equiv) of DMAP and 6 mL of anhydrous tetrahydrofuran were added into a 50 ml dry Schlenk tube. A reaction flask was placed in a cold trap (−10° C.). 0.88 mL (1.4 mmol) of an n-hexane solution of 1.6 M n-butyllithium was slowly added to carry out a reaction for 10 min, 0.77 mL. (1.4 mmol) of a tetrahydrofuran/n-heptane/ethylbenzene solution of 2.0 M LDA was added dropwise to carry out another reaction at a temperature of −10° C. for half an hour, and then. 8 ml of a THE solution of 262.6 mg (0.8 mmol, 1.0 equiv) of 3-benzyl-6-bromo-2-methoxyquinoline was slowly and completely added dropwise within 1 b. After the addition was completed, continuous stirring was performed for 1 h. Then, the temperature of the cold trap was lowered to −20° C., and a solution formed by 212.8 mg (0.96 mmol, 1.2 equiv) of 3-N,N-dimethylamino-1-naphthyl-1-propanone and 6 mL of THE was slowly added within next 60 min, wherein the temperature of a reaction system was maintained at −80° C. in the addition process. After the addition was completed, a continuous reaction was carried out for 4 h. After the reaction was completed, a reaction solution was quenched with a saturated ammonium chloride solution at low temperature, transferred to a place at room temperature and extracted with ethyl acetate (3×5 mL), followed by column chromatography to obtain a pure product with a yield of 35%. According to determination of a crude product by nuclear magnetic resonance, the product has a dr value of 1.5:1. According to determination by HPLC, the product has an ee value of 45%.

### Example 23

Under the protection of nitrogen, 1.3 mg (0.0096 mmol, 0.012 equiv) of zinc chloride, 470.6 mg (1.76 mmol, 2.2 equiv) of (1S,2K)-2-amino-1,2-di-mi-methylphen ylethanol (L8). 98 mg (0.8 mmol, 1.0 equiv) of DMAP and 6 ml, of anhydrous tetrahydrofuran were added into a 50 mL dry Schlenk tube. A reaction flask was placed in a cold trap (−10° C.), 0.88 ml (1.4 mmol) of an n-hexane solution of 1.6 M n-butyllithium was slowly added to carry out a reaction for 10 min, 0.5 ml. (1.0 mmol) of a tetrahydrofuran/n-heptane/ethylbenzene solution of 2.0 M LDA was added dropwise to carry out another reaction at a temperature of −10° C. for half an hour, and then, 8 mL of a THE solution of 262.6 mg (0.8 mmol, 1.0 equiv) of 3-benzyl-6-bromo-2-methoxyquinoline was slowly and completely added dropwise within 1 h. After the addition was completed, continuous stirring was performed for 1 h. Then, the temperature of the cold trap was lowered to −20° C., and a solution formed by 212.8 mg (0.96 mmol, 1.2 equiv) of 3-N,N-dimethylamino-1-naphthyl-1-propanone and 6 ml of THE was slowly added within next 60 min, wherein the temperature of a reaction system was maintained at −80° C. in the addition process. After the addition was completed, a continuous reaction was carried out for 4 h. After the reaction was completed, a reaction solution was quenched with a saturated ammonium chloride solution at low temperature, transferred to a place at room temperature and extracted with ethyl acetate (3×5 mL), followed by column chromatography to obtain a pure product with a yield of 33%. According to determination of a crude product by nuclear magnetic resonance, the product has a dr value of 2:1. According to determination by HPLC, the product has an ee value of 78%.

### Example 24

Under the protection of nitrogen, 141.6 mg (1.9 mmol, 2.4 equiv) of potassium chloride. 674.4 mg (2.6 mmol, 3.2 equiv) of (1S,2R)-2-amino-1,2-di-o-methoxyphenylethanol (19), 98 mg (0.8 mmol. 1.0 equiv) of DMAP and 6 ml of anhydrous tetrahydrofuran were added into a 50 mL dry Schlenk tube. A reaction flask was placed in a cold trap (−10° C.), 0.5 ml (0.8 mmol) of an n-hexane solution of 1.6 M n-butyllithium was slowly added to carry out a reaction for 10 min, 0.4 mL (0.8 mmol) of a tetrahydrofuran solution of 2.0 M potassium bis(trimethylsilyl)amide was added dropwise to carry out another reaction at a temperature of −10° C. for half an hour, and then, 8 mL of a THE solution of 262.6 mg (0.8 mmol, 1.0 equiv) of 3-benzyl-6-bromo-2-methoxyquinoline was slowly and completely added dropwise within 1 h. After the addition was completed, continuous stirring was performed for 1 h. Then, the temperature of the cold trap was lowered to −20° C., and a solution formed by 212.8 mg (0.96 mmol, 1.2 equiv) of 3-N,N-dimethylamino-1-naphthyl-1-propanone and 6 mL of THE was slowly added within next 60 min, wherein the temperature of a reaction system was maintained at −80° C. in the addition process. After the addition was completed, a continuous reaction was carried out for 4 h. After the reaction was completed, a reaction solution was quenched with a saturated ammonium chloride solution at low temperature, transferred to a place at room temperature and extracted with ethyl acetate (3×5 mL), followed by column chromatography to obtain a pure product with a yield of 37%. According to determination of a crude product by nuclear magnetic resonance, the product has a dr value of 1:1. According to determination by HPLC, the product has an ee value of 57%.

### Example 25

Under the protection of nitrogen, 70.1 mg (1.2 mmol, 1.5 equiv) of sodium chloride, 685.6 mg (3.2 mmol, 4.0 equiv) of (1S,2S)-1,2-diphenylethane-1,2-diol (L10), 98 mg (0.8 mmol, 1.0 equiv) of DMAP and 6 mL of anhydrous tetrahydrofuran were added into a 50 mL dry Schlenk tube. A reaction flask was placed in a cold trap (−10° C.), 0.88 ml. (1.4 mmol) of a tetrahydrofuran solution of 1.6 M tert-butyllithium was slowly added to carry out a reaction for 10 min, 0.8 mL. (1.6 mmol) of a tetrahydrofuran solution of 2.0 M sodium bis(trimethylsilyl)amide was added dropwise to carry out another reaction at a temperature of −10° C. for half an hour, and then, 8 mL of a THE solution of 262.6 mg (0.8 mmol. 1.0 equiv) of 3-benzyl-6-bromo-2-methoxyquinoline was slowly and completely added dropwise within 1 h. After the addition was completed, continuous stirring was performed for 1 h. Then, the temperature of the cold trap was lowered to −20° C., and a solution formed by 212.8 mg (0.96 mmol, 1.2 equiv) of 3-N,N-dimethylamino-1-naphthyl-1-propanone and 6 mL of toluene was slowly added within next 60 min, wherein the temperature of a reaction system was maintained at −80° C. in the addition process. After the addition was completed, a continuous reaction was carried out for 4 h. After the reaction was completed, a reaction solution was quenched with a saturated ammonium chloride solution at low temperature, transferred to a place at room temperature and extracted with ethyl acetate (3×5 ml), followed by column chromatography to obtain a pure product with a yield of 39%. According to determination of a crude product by nuclear magnetic resonance, the product has a dr value of 1.8:1. According to determination by HPLC, the product has an ee value of 61%.

### Example 26

Under the protection of nitrogen, 17.8 mg (0.16 mmol, 0.2 equiv) of calcium chloride, 639.2 mg (2.8 mmol, 3.5 equiv) of (18,28)-2-methoxy-1,2-diphenyl-1-ethanol (L11), 98 mg (0.8 mmol, 1.0 equiv) of DMAP and 6 ml of anhydrous tetrahydrofuran were added into a 50 mL dry Schlenk tube. A reaction flask was placed in a cold trap (−10° C.). 0.88 ml (1.4 mmol) of a tetrahydrofuran solution of 1.6 M sec-butyllithium was slowly added to carry out a reaction for 10 min, 1.3 mL. (2.6 mmol) of a tetrahydrofuran solution of 2.0 M lithium bis(trimethylsilyl)amide was added dropwise to carry out another reaction at a temperature of −10° C. for half an hour, and then, 8 mL of a THE solution of 262.6 mg (0.8 mmol, 1.0 equiv) of 3-benzyl-6-bromo-2-methoxyquinoline was slowly and completely added dropwise within 1 h. After the addition was completed, continuous stirring was performed for 1 h. Then, the temperature of the cold trap was lowered to −20° C. and a solution formed by 212.8 mg (0.96 mmol, 1.2 equiv) of 3-N,N-dimethylamino-1-naphthyl-1-propanone and 6 mL of ethylene glycol diethyl ether was slowly added within next 60 min, wherein the temperature of a reaction system was maintained at −80° C. in the addition process. After the addition was completed, a continuous reaction was carried out for 4 h. After the reaction was completed, a reaction solution was quenched with a saturated ammonium chloride solution at low temperature, transferred to a place at room temperature and extracted with ethyl acetate (3×5 mL), followed by column chromatography to obtain a pure product with a yield of 26%. According to determination of a crude product by nuclear magnetic resonance, the product has a dr value of 1:1. According to determination by HPLC, the product has an ee value of 32%.

### Example 27

Under the protection of nitrogen, 31.1 mg (0.24 mmol, 0.3 equiv) of anhydrous nickel chloride, 872.3 mg (3.6 mmol, 4.5 equiv) of (1S,2S)-1,2-dimethoxy-1,2-diphenylethane (L12), 440 μL. (4.0 mmol, 5.0 equiv) of DABCO and 6 mL of anhydrous tetrahydrofuran were added into a 50 mL dry Schlenk tube. A reaction flask was placed in a cold trap (−10° C.), 0.4 mL (0.8 mmol) of a tetrahydrofuran solution of 2.0 M lithium tetramethylpiperidine (LTMP) was slowly added to carry out a reaction at a temperature of −10° C. for half an hour, and then, 8 mL, of a THE solution of 262.6 mg (0.8 mmol, 1.0 equiv) of 3-benzyl-6-bromo-2-methoxyquinoline was slowly and completely added dropwise within 1 h. After the addition was completed, continuous stirring was performed for 1 h. Then, the temperature of the cold trap was lowered to −20° C., and a solution formed by 212.8 mg (0.96 mmol, 1.2 equiv) of 3-N,N-dimethylamino-1-naphthyl-1-propanone and 6 ml of 1,4-dioxane was slowly added within next 120 min, wherein the temperature of a reaction system was maintained at −80° C. in the addition process. After the addition was completed, a continuous reaction was carried out for 4 h. After the reaction was completed, a reaction solution was quenched with a saturated ammonium chloride solution at low temperature, transferred to a place at room temperature and extracted with ethyl acetate (3×5 mL), followed by column chromatography to obtain a pure product with a yield of 27%. According to determination of a crude product by nuclear magnetic resonance, the product has a dr value of 1:1. According to determination by HPLC, the product has an ce value of 32%.

The descriptions above are only some embodiments of the present invention. It is to be understood that the present invention is not limited to the above specific embodiments, and all equivalent changes and modifications made within the scope of the patent application for the present invention fall within the scope of the present invention.

What is claimed is:

1. A chiral bimetallic cooperative catalysis system for a synthesis of (1R,2S)-bedaquiline, wherein the chiral bimetallic cooperative catalysis system is formed by using a lithium, sodium or potassium salt in cooperation with metal salt and selecting a ligand and an additive.

2. The chiral bimetallic cooperative catalysis system according to claim 1, wherein the lithium, sodium or potassium salt is derived from one or a combination of n-butyllithium, sec-butyllithium, tert-butyllithium, lithium diisopropylamide, lithium tetramethylpiperidine, lithium bis(trimethylsilyl)amide, sodium bis(trimethylsilyl)amide, and potassium bis(trimethylsilyl)amide.

3. The chiral bimetallic cooperative catalysis system according to claim 1, wherein the metal salt is a metal halide and comprises one or more of MgCl₂, ZnCl₂, CuCl₂, AlCl₃, NiCl₂, CoCl₂, FeCl₃, NaCl, KCl, TiCl₄, CaCl₂, LiCl, LiBr, Lil, and LiF.

4. The chiral bimetallic cooperative catalysis system according to claim 1, wherein the ligand has a structural formula of 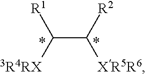
wherein X and X′ are separately selected from an N atom or an O atom; R¹, R², R³, R⁴, R⁵ and R⁶ are separately selected from H, C₁₋₁₆ straight-chain or branch-chain alkyl, aryl, or C₃₋₇ cycloalkyl, the aryl is phenyl substituted with R⁷, the R⁷ is H, C₁₋₁₆ alkyl, halogen, C₁₋₁₆ alkoxyl, or OCOR⁸, the R⁸ is C₁₋₆ alkyl, a substitution with the R⁷ refers to a single substitution or multiple substitutions, and the multiple substitutions are the same or different; and the R¹, the R², the R³, the R⁴, the R⁵ and the R⁶ are the same or different or two of the R¹ the R² the R³ the R⁴ the R⁵ and the R⁶ form at least one ring.

5. The chiral bimetallic cooperative catalysis system according to claim 4, wherein in the structural formula of the ligand, the R¹ and the R² are the same or different or form a first ring; the R³ and the R⁴ are the same or different or form a second ring; the R³ and the R¹ form a third ring or do not form the third ring; the R³ and the R⁶ are the same or different or form a fourth ring; the R⁵ and the R² form a fifth ring or do not form the fifth ring; and * refers to chirality with a configuration of RR, SS, RS or SR.

6. The chiral bimetallic cooperative catalysis system according to claim 1, wherein the additive is an organic alkali with a structural formula of 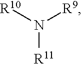
wherein R⁹, R¹⁰ and R¹¹ are separately H, C₁₋₆ straight-chain or branch-chain alkyl, aryl, or C₃₋₇ cycloalkyl, the aryl is phenyl substituted with R¹², and the R¹² is H, C₁₋₆ alkyl, halogen, C₁₋₆ alkoxyl, or OCOR¹³; the R¹³ is C₁₋₆ alkyl; a substitution with the R¹² refers to a single substitution or multiple substitutions, and the multiple substitutions are the same or different; and the R⁹, the R¹⁰ and the R¹¹ are the same or different or two of the R⁹ the R¹⁰ and the R¹¹ form a ring.

7. The chiral bimetallic cooperative catalysis system according to claim 6, wherein the organic alkali is selected from 1,8-diazabicyclo[5,4,0]undec-7-ene (DBU), 1,5-diazobicyclo[4,3,0]non-S-ene (DBN), N,N,N′,N′-tetramethylethylenediamine (TMEDA), hexamethylphosphoric triamide (HMPA), 4-dimethylaminopyridine (DMAP), triethylenediamine (DABCO), 2,2,6,6-tetramethylpiperidine (TMP), N,N-diisopropylamine (DIPA), pyridine, pyrrolidine, amantadine, Sparteine, and: BuNHSi (CH).

8. A method of synthesizing (1R,2S)-bedaquiline using the chiral bimetallic cooperative catalysis system according to claim 1, comprising: slowly adding an organic solution of 6-bromo-3-benzyl-2-methoxyquinoline (I) dropwise at a temperature of −78° C. to an organic solution of the chiral bimetallic cooperative catalysis system to carry out a first reaction, wherein the chiral bimetallic cooperative catalysis system is formed by a mixed reaction of the lithium, sodium or potassium salt, the metal salt, the ligand, and the additive, then continuously adding an organic solution of 3-N,N-dimethylamino-1-naphthyl-1-propanone (II) to carry out a second reaction, and subjecting a resulting product to a separation and a purification to obtain the (1R,2S)-bedaquiline.

9. The method according to claim 8, wherein a use amount of the lithium, sodium or potassium salt is 1-5 times of an equivalent of the 6-bromo-3-benzyl-2-methoxyquinoline (1); a use amount of the metal salt is 0.01-2.0 times of the equivalent of the 3-dimethylamino-1-naphthyl-1-propanone (II); a molar ratio of the metal salt to the ligand is 1.0: (0.1-5.0); and a use amount of the additive is 0.1-20 times of the equivalent of the 6-bromo-3-benzyl-2-methoxyquinoline (I).

10. The method according to claim 8, wherein an organic solvent used in each of the organic solution of the 6-bromo-3-benzyl-2-methoxyquinoline (1), the organic solution of chiral bimetallic cooperative catalysis system, and the organic solution of the 3-NA-dimethylamino-1-naphthyl-1-propanone (II) is selected from one or a mixture of ethyl ether, tetrahydrofuran, methyl tert-butyl ether, 1,4-dioxane, diisopropyl ether, ethylene glycol dimethyl ether, toluene, xylene, ethylbenzene, n-hexane, cyclohexane, n-heptane, and n-pentane.

11. The method according to claim 8, wherein the lithium, sodium or potassium salt is derived from one or a combination of n-butyllithium, sec-butyllithium, tert-butyllithium, lithium diisopropylamide, lithium tetramethylpiperidine, lithium bis(trimethylsilyl)amide, sodium bis(trimethylsilyl)amide, and potassium bis(trimethylsilyl)amide.

12. The method according to claim 8, wherein the metal salt is a metal halide and comprises one or more of MgCl₂, ZnCl₂, CuCl₂, AlCl₃, NiCl₂, CoCl₂, FeCl₃, NaCl, KCl, TICl₄, CaCl₂, LiCl, LiBr, Lil, and LiF.

13. The method according to claim 8, wherein the ligand has a structural formula of 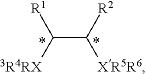
wherein X and X′ are separately selected from an N atom or an O atom; R¹, R², R³, R⁴, R⁵ and R⁶ are separately selected from H, C₁₋₁₆ straight-chain or branch-chain alkyl, aryl, or C₃₋₇ cycloalkyl, the aryl is phenyl substituted with R⁷, the R⁷ is H, C₁₋₈ alkyl, halogen, C₁₋₆ alkoxyl, or OCOR⁸, the R⁸ is C₁₋₆ alkyl, a substitution with the R⁷ refers to a single substitution or multiple substitutions, and the multiple substitutions are the same or different; and the R¹, the R², the R³, the R⁴, the R⁵ and the R⁶ are the same or different or two of the R¹, the R², the R³, the R⁴, the R⁵ and the R⁶ form at least one ring.

14. The method according to claim 13, wherein in the structural formula of the ligand, the R¹ and the R² are the same or different or form a first ring; the R³ and the R⁴ are the same or different or form a second ring; the R³ and the R⁴ form a third ring or do not form the third ring; the R⁵ and the R⁶ are the same or different or form a fourth ring; the R⁵ and the R² form a fifth ring or do not form the fifth ring; and * refers to chirality with a configuration of RR, SS, RS or SR.

15. The method according to claim 8, wherein the additive is an organic alkali with a structural formula of 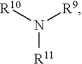
wherein R⁹, R¹⁰ and R¹¹ are separately H, C₁₋₆ straight-chain or branch-chain alkyl, aryl, or C₃₋₇ cycloalkyl, the aryl is phenyl substituted with R¹², and the R¹² is H, C₁₋₆ alkyl, halogen, C₁₋₆ alkoxyl, or OCOR¹³; the R¹³ is C₁₋₆ alkyl; a substitution with the R¹² refers to a single substitution or multiple substitutions, and the multiple substitutions are the same or different; and the R⁹, the R¹⁰ and the R¹¹ are the same or different or two of the R⁹, the R¹⁰ and the R¹¹ form a ring.

16. The method according to claim 15, wherein the organic alkali is selected from 1,8-diazabicyclo[5,4,0]undec-7-ene (DBU), 1,5-diazobicyclo[4.3.0]non-5-ene (DBN), N,N,N′,N′-tetramethylethylenediamine (TMEDA), hexamethylphosphoric triamide (HMPA), 4-dimethylaminopyridine (DMAP), triethylenediamine (DABCO), 2,2,6,6-tetramethylpiperidine (TMP), N,N-diisopropylamine (DIPA), pyridine, pyrrolidine, amantadine, Sparteine, and/BuNHSi (CH)₃.
